(19) United States
(12) Patent Application Publication
(10) Pub. No.: US 2025/0090372 A1
(43) Pub. Date: Mar. 20, 2025

(54) MULTIDOSE DISPENSER CONTAINING A VISCOUS OPHTHALMIC PRODUCT

(71) Applicant: LABORATOIRES THEA, CLERMONT-FERRAND (FR)

(72) Inventor: Benjamin QUAGLIA, Châteaugay (FR)

(21) Appl. No.: 18/727,576

(22) PCT Filed: Jan. 5, 2023
(86) PCT No.: PCT/FR2023/050012

§ 371 Date: Jul. 9, 2024

(30) Foreign Application Priority Data
Jan. 10, 2022 (FR) 2200158

(51) Int. Cl. A61F 9/00

(52) U.S. Cl. CPC A61F 9/0008

(57) ABSTRACT

A dispenser containing a viscous ophthalmic product that is sterile and free from preservatives, including a reservoir having a wall suitable for deforming when pressure is applied to the reservoir by a user of the dispenser and for spontaneously returning to its original shape upon release. The reservoir further includes a flexible bag in the internal space defined by the wall thereof in which the viscous ophthalmic product is contained. The dispenser includes an air inlet means for receiving air between the wall of the reservoir and the flexible bag and an attached head which comprises a drip end piece having a dispensing opening, a unidirectional valve-interposed between the flexible bag and the dispensing opening, the valve being configured to prevent air from outside the dispenser from entering into the flexible bag.

The present invention relates to a multidose dispenser which contains a viscous ophthalmic product that is sterile and free from preservatives, and which is suitable for packaging, storing and drip dispensing the viscous ophthalmic product contained therein.

In the field of pharmaceutical compositions and in particular ophthalmic products, there are numerous products intended to be dripped on the surface of the eye. These products are often liquid. However, the use of viscous ophthalmic products is growing. Indeed, certain ophthalmic compositions prove to be more effective when delivered in the form of a viscous fluid, they cover the surface of the eye better and/or remain in contact with the eye longer, which improves the effectiveness of the product and local tolerance to this product.

“Viscous” (in particular in the expressions “viscous product”, “viscous fluid”, “viscous compositions”, gels, etc.) means that the viscosity is greater than or equal to 200 cP. The viscous ophthalmic products targeted in the context of the present invention are generally considered with a viscosity greater than 500 cP, and up to 1500 cP, in order to be administered in the form of drips.

The products considered viscous in this document can be Newtonian or non-Newtonian. The values indicated are obtained using the rotary viscometer method as defined in the European Pharmacopoeia 6.0 (01/2008).

Ophthalmic products are used in small doses, one or a few drips from a dispenser during each use, while the rest of the product contained in the dispenser must be kept for a certain time, generally several days or even several weeks. The product contained in the dispenser must generally be kept free from any environmental and/or microbiological contamination by bacterial or other agents coming from the outside of the dispenser.

There is also a particular interest in offering preservative-free ophthalmic solutions. Indeed, products used as a preservative in ophthalmic solutions, to ensure the sterility of ophthalmic products with respect to bacteria and fungi, can have significant adverse effects.

Preservatives, in particular benzalkonium chloride, are irritating to the eyes and can alter the tear film, and can cause tissue damage through apoptosis. Long-term use of preservatives has undesirable consequences on the eye.

Thus, the use of ophthalmic products that are free from preservatives is often recommended.

A first solution for maintaining the sterility of such preservative-free ophthalmic products consists of packaging them in n single doses, also called “unidoses”. Unidose packaging is, however, not applicable to all products, and generates a lot of waste, with the packaging of each dose having to be thrown away after use. For long-term treatment, unidose packaging does not appear appropriate.

In this context, dispensers containing several doses, or “multidose” dispensers, configured to ensure the sterility of the product they contain, including after opening and delivery of a first dose, have been developed. The purpose of these dispensers is to maintain sterility in the dispenser for as long as the consumption of the product lasts until the contents of the dispenser are completely exhausted.

For example, document FR2816600 discloses a drip type dispenser, the reservoir of which has a deformable wall allowing to create an excess pressure causing the passage of the product contained through a sterilising membrane. The surface of this membrane is partially hydrophilic and partially hydrophobic, and more particularly selectively permeable to aqueous liquids in its hydrophilic part and selectively permeable to air in its hydrophobic part. Thus, the liquid passes through the membrane in its hydrophilic part when dispensed before reaching a drip dispensing end piece, while air can be drawn into the dispenser through the hydrophobic part to compensate for the volume of liquid dispensed when the reservoir is no longer compressed and returns to its initial shape. A porous buffer allows regulation of the flow in the dispenser, necessary for the proper formation of drips.

This type of dispenser is fully satisfactory for packaging and dispensing many sterile preservative-free products, and in particular preservative-free liquid ophthalmic products.

However, this dispenser is not suitable for dispensing certain products. In particular, viscous ophthalmic products cannot be dispensed satisfactorily by this type of dispenser. Indeed, due to the low porosity of the membrane, viscous products cannot, or only poorly pass therethrough. To pass through the membrane, the more viscous the product, the more pressure must be applied on the product. The risk of rupture of the membrane is then significant. For the most viscous products, dispensing is simply impossible.

Generally speaking, when a viscous product is packaged in a multidose dispenser with a deformable wall, the viscous product tends to adhere to the walls and the bottom of the dispenser. In particular, between two dispensings, the product falls to the bottom of the dispenser, and is no longer in contact with the dispensing end piece. During the next dispensing of the product, the dispenser is turned over but the product adheres to the bottom of the dispenser and does not immediately contact the dispensing end piece. The deformation of the wall thus firstly results in the expulsion of the air present in the dispenser, and, as dispensing progresses, it is necessary to deform the dispenser more and more to cause a dispensing of product, which can also damage the dispenser. The adhesion of the product to the walls of the dispenser makes it complex or even impossible to deliver all the product present in the dispenser, which is not desirable for an ophthalmic product, which is packaged to guarantee a precise number of drips and which generally has great value.

Document EP1985543 presents a dispenser for an ophthalmic product. The dispenser reservoir is of the “delamination” type, which means that the internal layer of the reservoir can be separated from the rest of the walls to form a deformable internal pocket. This dispenser includes a non-return valve through which the product leaving the dispenser passes and includes, downstream of this valve, a filter allowing the filtering of certain bacteria. This dispenser is, however, not suitable for delivering a viscous product and/or guaranteeing the sterility of the product delivered.

Document EP1593605 has a similar teaching.

The present invention aims at providing a multidose dispenser, containing a viscous ophthalmic product that is sterile and free from preservatives, which overcomes all or part of the problems mentioned above.

Thus, the invention relates to a dispenser containing a viscous ophthalmic product.

As indicated above, these are products having a viscosity greater than or equal to 200 cP. The dispenser includes a reservoir having a wall defining an internal space and which is suitable for deforming when pressure is applied to the reservoir by a user of the dispenser and for spontaneously returning to its original shape upon release of said pressure. The viscous ophthalmic product contained in the dispenser is sterile and free from preservatives. The reservoir further includes a flexible bag in the internal space defined by the wall. The flexible bag contains the viscous ophthalmic product.

The dispenser further includes:
an air inlet means for receiving air between the wall of the reservoir and the flexible bag,
a head attached onto a neck of the reservoir and including a drip end piece having a dispensing opening, and
a unidirectional valve interposed between the reservoir and the dispensing opening, the unidirectional valve being configured to prevent air from outside the dispenser from entering into the reservoir. Thus, once the viscous ophthalmic product has been dispensed, the dispensed volume of ophthalmic product is compensated by a corresponding volume of air being received between the wall of the reservoir and the flexible bag via the air inlet means.

The dispenser includes internal surfaces located between the unidirectional valve and the dispensing opening, and all these internal surfaces having a bactericidal treatment and/or a treatment that reduces the wettability thereof.

“All internal surfaces” means in particular all the surfaces exposed to the ophthalmic product when it leaves the dispenser.

The present invention thus provides a drip dispenser containing a viscous ophthalmic product, which is perfectly suited to the dispensing of such a product. The viscosity of the product contained in the dispenser is measured according to the rotating viscometer method as defined in the European Pharmacopoeia 6.0 (01/2008). It is notable that this method has not evolved to date in the European Pharmacopoeia, it is therefore possible to refer to the European Pharmacopoeia from version 6.0 to version 10.6 in force to date.

The proposed dispenser is easy to use for the user, in that it allows the dispensing of product by simply pressing on the reservoir, which is intuitive.

Furthermore, the flexible bag which is present in the internal space defined by the wall of the reservoir allows complete dispensing of the product it contains, that is to say that all doses of product contained in the flexible bag can be actually dispensed.

Product dispensing can also be carried out while the dispenser is positioned in various orientations. Indeed, as doses of product are dispensed, the volume of product dispensed is compensated by the entry of an equivalent volume of air between the deformable wall of the reservoir and the flexible bag. The volume of the flexible bag adapts to that of the product it contains. The product contained in the flexible bag thus remains in the immediate vicinity of the dispensing head, and no air bubbles form in the flexible bag. This is particularly important since the product contained in the dispenser is viscous, and therefore likely to adhere to the walls of the dispenser. The implementation of a flexible bag in the reservoir of the dispenser thus allows the immediate delivery of the product, and also allows the total or almost total delivery of the product contained in the dispenser.

When the user applies pressure on the deformable wall, the air present between the deformable wall of the reservoir and the flexible bag is prevented from escaping from this space (whether by a means which automatically prevents this escape of the air or by an action of the user), so that the flexible bag is itself pressurised, causing the expulsion of the product through the drip end piece.

Furthermore, in order to make the dispenser suitable for dispensing a viscous ophthalmic product that is free from preservatives, the surfaces of the dispenser likely to be in contact with the product and which are located downstream of the unidirectional valve having a bactericidal treatment and/or a treatment that reduces the wettability thereof.

Throughout this document, the terms “upstream” and “downstream” are understood by considering the direction of flow of the viscous ophthalmic product during its delivery, that is to say from the reservoir until leaving the end piece of the dispenser.

A bactericidal treatment of the internal surfaces located downstream of the unidirectional valve and/or a treatment that reduces the wettability of these surfaces allows, in combination with the other features of the dispenser, to package the ophthalmic product free from preservatives. Indeed, after delivery, any return of product to the reservoir is prevented by the unidirectional valve. This helps maintain the sterility of the product present in the reservoir. A remainder of product may nevertheless remain between the unidirectional valve and the dispensing opening of the dispenser. The bactericidal treatment prevents any bacterial development in this remaining product, which, even if it is a very small amount, will be expelled from the dispenser during dispensing. Moreover, the the next treatment prevents any bacterial development on the internal surfaces of the dispenser which have the treatment.

In addition or alternatively, a treatment to limit wettability allows to limit any remaining ophthalmic product, after dispensing, downstream of the unidirectional valve of the dispenser. The presence of such a remainder is all the more probable, and the remainder all the more significant, as the ophthalmic product is viscous. It is recalled that wettability can be defined as the ability of a surface to be wetted by a liquid. It can be measured by the contact angle of a drip of a liquid placed on the considered surface. Low wettability corresponds to a situation wherein the product tends to flow easily over the surface, and does not tend to adhere thereto. Such a coating therefore allows to limit the risk the of bacterial development downstream of unidirectional valve, and also to guarantee better reproducibility of the doses delivered by the dispenser, despite the viscous nature of the product.

The dispenser containing a viscous ophthalmic product that is sterile and free from preservatives thus responds, through an appropriate combination of features, to the dual problem of maintaining the sterility of the product and easy delivery.

The flexible bag can be formed by delamination of an internal layer of the reservoir. As an alternative to a flexible bag present from manufacturing in the dispenser reservoir, a flexible bag obtained by delamination of the wall of the reservoir is a simple, industrially mastered, and inexpensive solution to obtain the function sought in the invention.

Alternatively, the flexible bag may be a retractable flexible bag, which is attached. The use of such a retractable flexible bag allows in particular to select a bag having a nominal capacity corresponding precisely to the initial volume of viscous ophthalmic product to be packaged, while using the same deformable wall for different volumes of product to be packaged.

The air inlet means can be formed by a hole in the wall of the reservoir adapted to be closed by a finger of the user.

This is a particularly simple solution to prevent the air contained between the wall of the reservoir and the flexible bag from escaping when dispensing the product to the outside of the reservoir, that is to say when pressure is applied to the reservoir. Appropriate positioning of the hole formed in the wall makes using the dispenser perfectly intuitive. The hole can thus be advantageously positioned in an area of the reservoir where a finger of the user naturally rests when holding the dispenser in order to dispense the product contained in the dispenser. The hole can thus be positioned on a side wall of the reservoir, in particular in the vicinity of the neck of the reservoir, or, alternatively, on the bottom of the reservoir.

Alternatively, the air inlet means may include a unidirectional valve, configured to allow receiving air between the wall of the reservoir and the flexible bag and to prevent the air present between the wall of the reservoir and the flexible bag from escaping to the outside of the reservoir.

According to this configuration, the escape of air towards the outside of the reservoir is automatically prevented, which makes the use of the reservoir extremely simple, because the user does not have to do any particular action (however simple it may be) compared to a conventional dispenser with a deformable reservoir.

The bactericidal treatment can be based on silver ions or zinc ions.

The bactericidal treatment may include silver ions or zinc ions included in the mass of the elements forming said internal surfaces.

Alternatively or in addition, the bactericidal treatment may include a bactericidal surface coating.

This type of bactericidal treatment is perfectly suited to ophthalmic products, intended to be applied to a patient's eye.

Of course, bactericidal treatment can also have an antifungal effect.

The treatment that reduces the wettability of the internal surfaces located between the unidirectional valve and the dispensing opening can be a treatment of the fluorinated or silicone plasma treatment type.

The treatment that reduces the wettability of the internal surfaces located between the unidirectional valve and the dispensing opening may be laser surface texturing.

The dispenser containing a viscous ophthalmic product may include a removable cap, attached to the dispensing head.

The cap ensures an airtight closure of the dispenser. Thus it helps to guarantee that the sterility of the product present in the reservoir is maintained. It also guarantees the protection of the dispensing end piece, in particular against possible contaminants (dust, bacteria, etc.) between two dispensings of doses of the ophthalmic product. Any remaining product downstream of the unidirectional valve of the dispenser is thus protected.

The unidirectional valve may include an element deformable under the effect of excess pressure inside the reservoir of the dispenser, or include a ball check-valve.

The viscous ophthalmic product contained in the dispenser may contain one or more active ingredients in a therapeutically effective amount selected from the group of anti-glaucoma, anti-inflammatoi immunosuppressants, antivirals, antibacterials, antifungals or antioxidants. The viscous ophthalmic product contained in the dispenser may include one or more active ingredients in a therapeutically effective amount against dry eyes such as polyacrylic acid derivatives, carbomers, trehalose, hyaluronic acid or its salts, povidone, polyvinyl alcohol, cellulose derivatives, chitosan derivatives, phospholipids, triglycerides.

Active means a compound administered to a subject having the symptoms (signs) of a pathology with the aim of reducing or eliminating these symptoms. The term “therapeutically effective amount” refers to an amount of active ingredient that is sufficient or effective to prevent or treat a disease or disorder including alleviating the symptoms of that disease or disorder.

Other features and advantages of the invention will appear in the description below.

In the appended drawings, given as non-limiting examples:

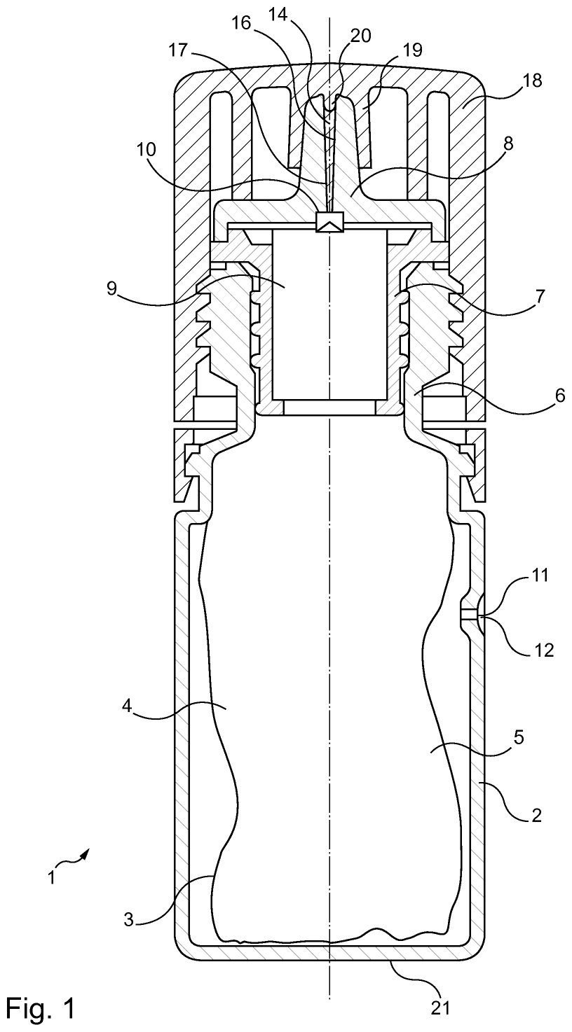

FIG. 1 shows, in a sectional view, a dispenser in accordance with one embodiment of the invention;

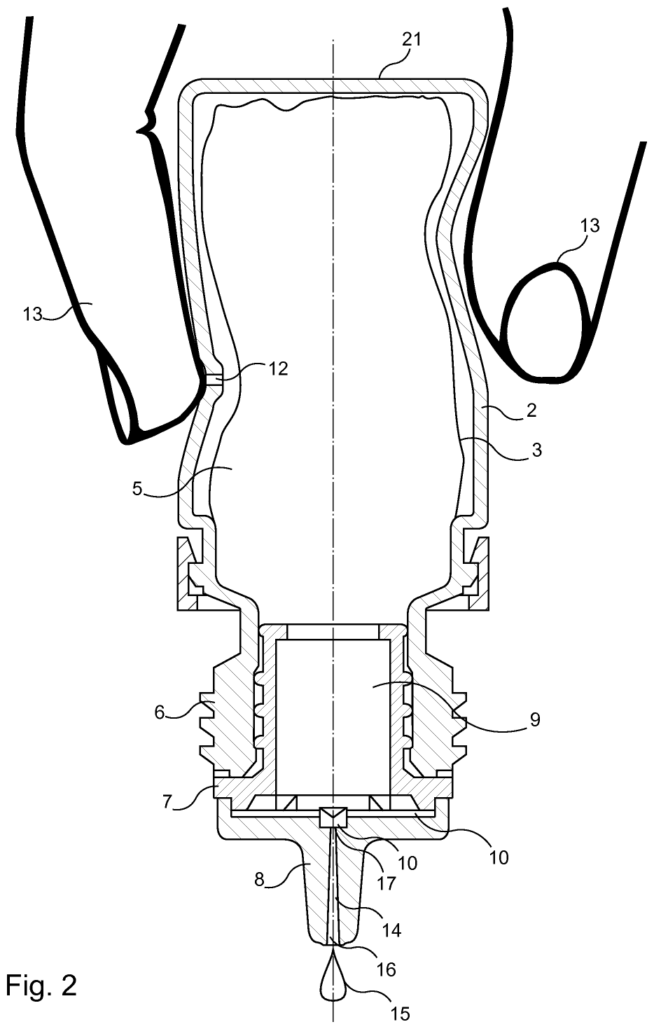

FIG. 2 shows, in a sectional view, the dispenser of FIG. 1 during delivery of the product it contains;

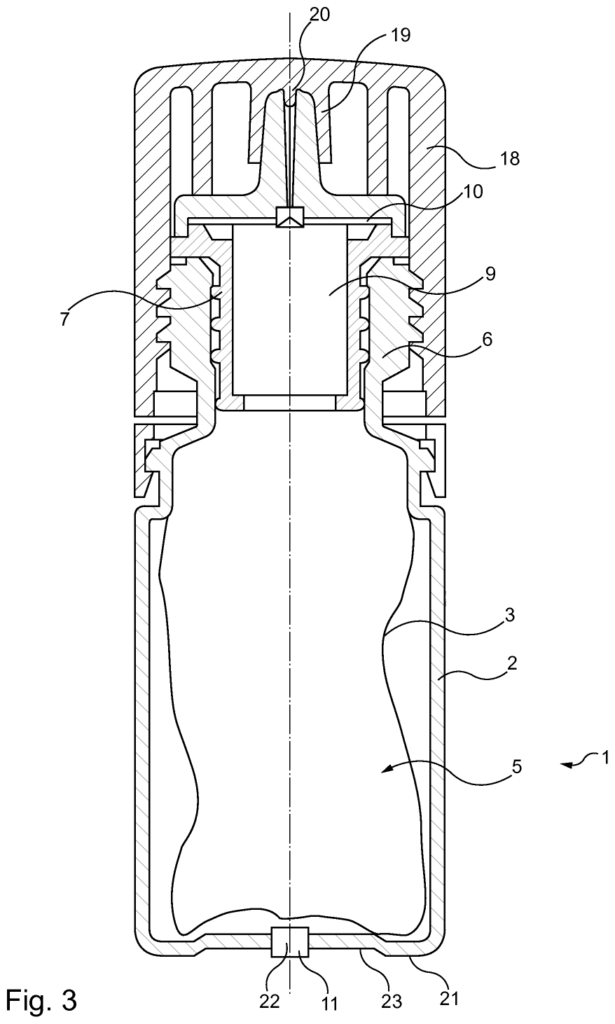

FIG. 3 shows, in a sectional view, a dispenser in accordance with one embodiment the of invention, illustrating several aspects that can be implemented in such a dispenser;

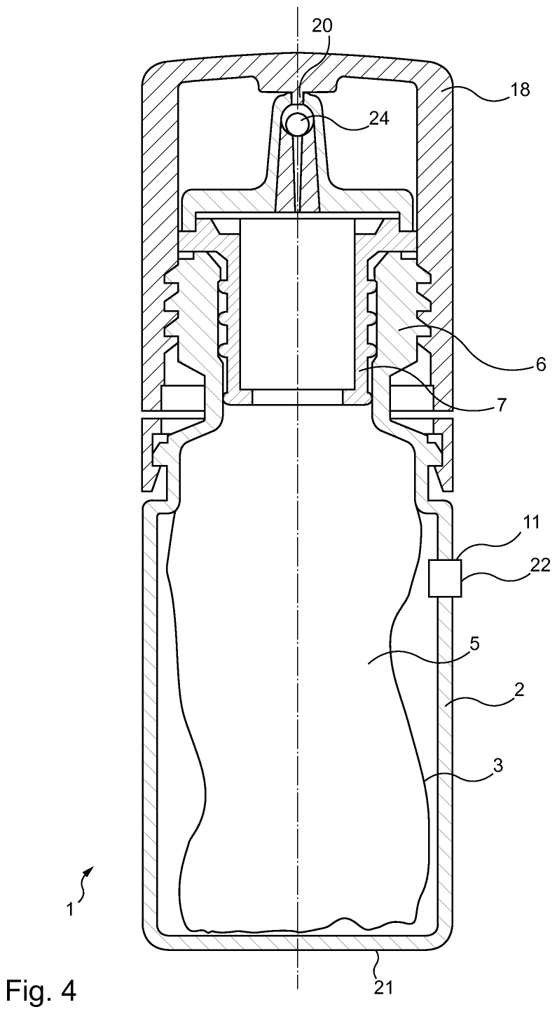

FIG. 4 shows, in a sectional view, a dispenser in accordance with another embodiment of the invention, illustrating several aspects that can be implemented in such a dispenser;

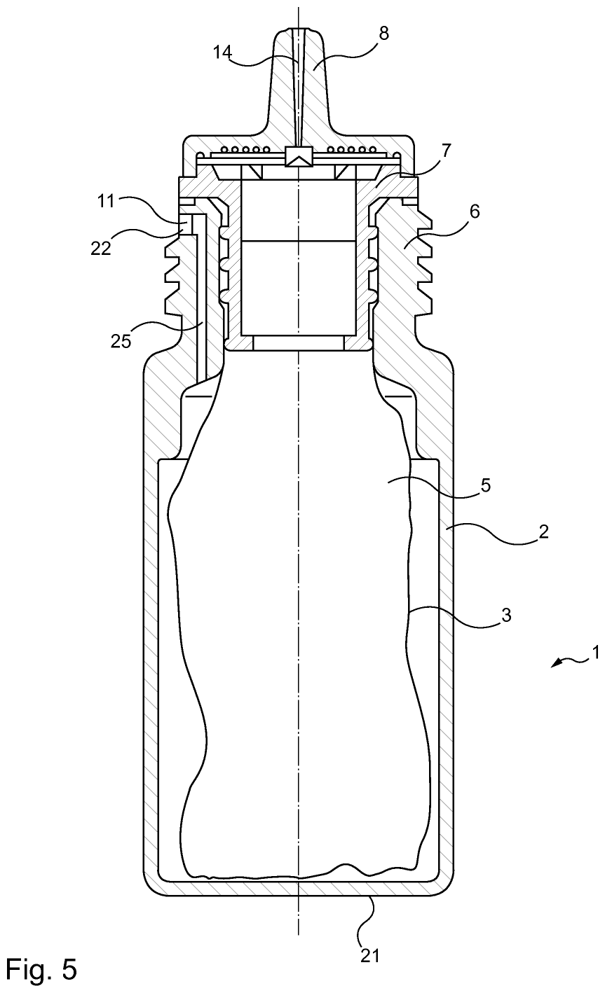

FIG. 5 shows, in a sectional view, a dispenser in accordance with another embodiment of the invention;

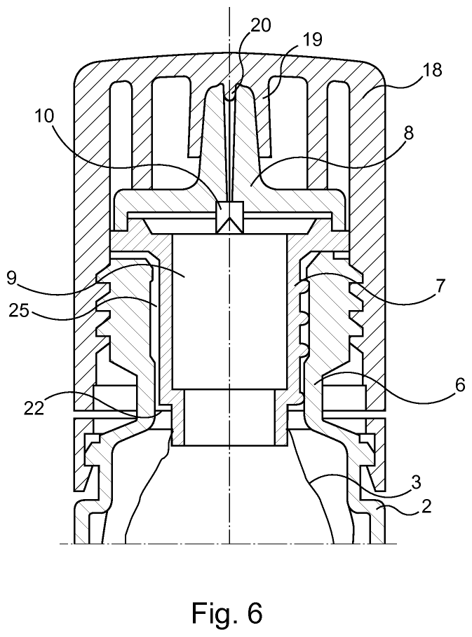

FIG. 6 shows, in a partial sectional view, a dispenser in accordance with yet another embodiment of the invention.

FIG. 1 shows a dispenser containing a viscous ophthalmic product according to a first embodiment of the invention.

The reservoir 1 includes a deformable wall 2, and which forms an internal space of the reservoir 1. The wall 2 is in particular configured to be able to be deformed under pressure exerted by the hand of a user when the latter wishes to carry out the delivery of one or more drips of product.

In the example shown, the wall 2 is a cylindrical peripheral wall. The wall 2 is elastically deformable, that is to say it spontaneously tends to return to its initial shape after pressure has ceased to be exerted thereon.

The reservoir includes, in its internal space, a flexible bag 3. The flexible bag 3 can be an attached pocket, formed from a flexible plastic material, silicone, etc. Alternatively, the flexible bag 3 is formed by delamination of the internal surface of the wall 2, that is to say that the flexible bag 3 detaches from the wall 2 during the first deliveries of product. The flexible bag forms a receiving space 4 wherein the viscous ophthalmic product is contained.

The flexible bag 3 of the reservoir 1 contains a viscous ophthalmic product 5, that is sterile and free from preservatives.

The flexible bag must therefore have sufficient permeability features to ensure good conservation of the product it contains over a long period, typically several weeks or several months.

In the example shown in FIG. 1, the flexible bag remains in contact with the wall 2 of the reservoir at least near a neck 6 of the reservoir.

A dispensing head is attached to the dispenser. In the exemplary embodiment shown here, the dispensing head of the dripping viscous ophthalmic product comprises an insert 7, disposed inside the neck 6 of the dispenser. An end piece 8 (or nozzle) for drip dispensing is fixed to (or formed by) the insert 7. The insert 7 is rigidly and sealingly fixed in the neck 6. The insert 7 can for example be forcibly inserted into the neck 6.

The insert 7 is said to be hollow, in that it provides a space 9 allowing the passage of the viscous ophthalmic product from the flexible bag 3 towards the end piece 8. The dispenser also includes a unidirectional valve 10.

The unidirectional valve is configured, in particular oriented, so as to allow the viscous ophthalmic product 5 to pass therethrough under the effect of an excess pressure inside the reservoir 1 compared to the outside (at atmospheric pressure).

In the example shown, this unidirectional valve 10 is interposed in the passage provided for the product, at the inlet of the dispensing nozzle 8.

The unidirectional valve shown here as an example has a lip that can be deformed under the effect of excess pressure in the reservoir 1 of the dispenser. At rest, in the absence of pressure difference on either side of the unidirectional valve 10, the latter is hermetically closed, and any depression in the reservoir tends to close the unidirectional valve even more strongly.

The dispenser further includes an air inlet means 11. The air inlet means 11 allows the air to pass through the wall 2 of the dispenser 1, so as to take place between said wall 2 and the flexible bag 3. In the example shown here, the air inlet means 11 is formed by a hole 12, for example a round hole a few millimetres in diameter.

The hole 12 can be formed in a slight recess in the wall 2.

FIG. 2 shows the dispenser of FIG. 1 when delivering the product contained therein. To cause the delivery of one or more drips of viscous ophthalmic product, a user presses the dispenser for example between two fingers 13, thus deforming the wall 2, and reducing its internal space. During manual compression of the dispenser, one of the fingers 13 is placed so as to close the hole 12, which prevents the air present between the wall 2 and the flexible bag 3 from escaping. This results in an increase in the pressure in the reservoir, both for the air present between the wall 2 and the flexible bag 3 and for the viscous ophthalmic product, the pressure being transmitted by the flexible bag 3.

The pressure difference between the internal space of the reservoir 1 and the ambient air causes a flow of viscous ophthalmic product contained in the reservoir through the unidirectional valve 10. The viscous ophthalmic product freely passes therethrough under the effect of the pressure generated by the force applied to the walls of the dispenser by the user, due to the orientation of the unidirectional valve 10.

The viscous ophthalmic product is then evacuated through the end piece 8, for example via a channel 14 of small section formed in the end piece 8 and allowing the formation of regular drips 15.

When the user releases the pressure he exerts on the reservoir 1, his finger 13 stops sealing the hole 12. The air is thus free to enter the space formed between the wall 2 and the flexible bag 3. At the same time, the unidirectional valve 10 opposes the re-aspiration of air into the flexible bag 3 via the end piece 8. As a result, the volume of viscous ophthalmic product expelled will be totally compensated by a corresponding volume of air being received into the reservoir 1 between the wall 2 and the flexible bag 3.

Nevertheless, and in particular to the extent that the product delivered is viscous, a remainder of product can adhere to the internal surfaces located downstream of the unidirectional valve 10. These surfaces include the surfaces 16 of the channel 14, as well as the external surface 17 of the unidirectional valve 10. The surface called external surface of the unidirectional valve 10 is its surface oriented towards the dispensing opening of the dispenser, that is to say the surface located at the base of the channel 14. More generally, these surfaces correspond to surfaces likely to be in contact with the viscous ophthalmic product when dispensed, and which may also be in contact with the air outside the dispenser, at least in a situation of use of the dispenser (typically when dispensing the viscous ophthalmic product).

The unidirectional valve 10 can advantageously be positioned at the end of the nozzle 8, thus minimising the amount of residual liquid downstream of the unidirectional valve. However, these internal surfaces downstream of the unidirectional valve cannot be completely eliminated because they participate in the formation of regular drips.

These internal surfaces located downstream of the unidirectional valve 10 are treated, and have a bactericidal treatment and/or a treatment that reduces their wettability.

These two types of treatment can, alternatively or in combination, make the dispenser suitable for delivering a viscous ophthalmic product that is sterile, free from preservatives.

Indeed, the area wherein these surfaces are present and where a residue of product is likely to remain after dispensing a dose of viscous ophthalmic product is not protected, at least during the dispensing of the product, against the arrival of contaminants (dust, bacteria) from outside the dispenser. Thus, a bactericidal treatment, for example a surface treatment at the surfaces 16 of the channel 14 and the external surface 17 of the unidirectional valve 10 and/or in the mass of the end piece 8 and/or the unidirectional valve 10, allows to guarantee the absence of bacterial proliferation on these surfaces, as well as, where applicable, in the remaining product which would be in contact with these surfaces.

A treatment which limits the wettability of these surfaces has the effect of limiting or avoiding the residual product downstream of the unidirectional valve 10. The question of excessive bacterial proliferation in a residual product is thus avoided. Ideally, the bactericidal treatment and the treatment which limits the wettability thereof are combined, so that the presence of residue is limited by the treatment which limits the wettability thereof while any bacterial or fungal development is avoided on the treated surfaces.

The dispenser proposed according to the present invention thus includes a combination of features which make it suitable for packaging and delivering multiple doses of the sterile and preservative-free viscous ophthalmic product contained therein.

Many other aspects that can be implemented on such a dispenser are illustrated in the following figures.

In order to further guarantee the absence of air suction in the flexible bag 3 and more generally in contact with the viscous ophthalmic product upstream of the unidirectional valve 10, various means can be used as alternatives or in addition to each other. Firstly, a removable cap 18 for closing the end piece 8 can be used. The cap 18 can in particular be adapted to be screwed around the neck 6 of the dispenser. The cap ensures airtightness thanks to its mode of fixing to the neck 6 of the dispenser, and/or thanks to complementary means. In particular, a receiving cylinder 19 of the end piece 8 can be formed in the cap 18, as well as a pin 20 for closing the channel 14. Thus, once the dispenser is closed, between two dispensings of product, no entry of air via the dispensing nozzle 8 can occur. Moreover, the cap 18 also provides hermetic protection of the internal part of the dispenser located downstream of the unidirectional valve 10, and in particular protection of the channel 14.

In the exemplary embodiment shown in FIGS. 1 and 2, the air inlet means 11 is formed of a hole 12 which can be easily closed by the user. The positioning of the hole 12, although it must be natural for the user, can however be adapted depending on the dispenser. It can be in the upper part of the wall 2, in the middle (according to the general direction of extension of the dispenser), or else on the bottom 21 of the dispenser.

As illustrated in FIG. 3 and FIG. 4, the hole 12 can be replaced by a unidirectional valve 22, allowing air to enter the space between the wall 2 and the flexible bag 3 but preventing it from coming out of this space. The unidirectional valve 22 can be a soft lip valve, a ball check-valve, etc. It can be located on the wall 2 as shown in FIG. 4 or on the bottom 21 of the dispenser as shown in FIG. 3. In the embodiment of FIG. 3, the unidirectional valve 22 is placed in a recess 23 formed in the bottom 1 of the dispenser.

In the example shown in FIG. 4, the unidirectional valve which allows the dispensing of the product and prevents the return of air into the reservoir is a ball check-valve 24. The ball check-valve 24 offers the same functionality as the unidirectional deformable lip valve described previously. The ball then advantageously carries (as well as the surfaces on the housing where it is located) the bactericidal treatment and/or treatment limiting wettability.

In the example shown in FIG. 4, the ball check-valve is formed in the vicinity of the dispensing opening of the viscous ophthalmic product, that is to say the outlet of the channel 14. This limits the internal surfaces located downstream of the unidirectional valve. This arrangement can obviously be adopted with any unidirectional valve technology. However, it can limit the regularity of the drips from the dispenser. FIG. 5 shows an embodiment wherein the air inlet means 11 is integrated into the upper part of the dispenser, namely in the example shown in its neck 6. A conduit 25 allows to guide the air entering through the air inlet means towards the space located between the wall 2 and the flexible bag 3. A unidirectional valve 22, forming air inlet means 11, is disposed at the mouth of the conduit 25. This embodiment allows discreet integration of the air inlet means and offers a positioning for this means which avoids any risk of involuntary blocking during the dispensing of the product contained in the dispenser.

Alternatively to the unidirectional valve, the air inlet means 11 can be equipped (in any embodiment of the invention) with an element permeable to air but adapted to generate a significant pressure loss (so that the diffusion of air through this element can only be done slowly). A thin air-permeable cover, for example made of silicone, can be used to form this element.

Thus, when delivering the product, when the user presses the reservoir 1, a significant pressure differential is created but the resistance against the expulsion of the product by the unidirectional valve 10 and the nozzle 8 is less than the resistance to the passage of air generated by the cover, and the time required for dispensing is too short to allow significant passage of air through the cover.

When the pressure on the reservoir is released, the entry of air through the nozzle 8 being prevented by the unidirectional valve 10, the pressure difference between the internal space of the reservoir and the outside causes the gradual passage of air through the cover, until the pressures are balanced. The compensation of the volume of dispensed product by air between the wall 2 and the flexible bag 3 is thus achieved. FIG. 6 shows another embodiment of the invention, wherein the flexible bag 3 is an attached flexible bag and the air inlet means includes an element forming a unidirectional valve 22 at the interface between the flexible bag and the element of the dispenser to which the flexible bag is connected.

In the example shown here, the flexible bag 3 is connected to the insert 7 (this configuration being also applicable to any embodiment including an attached pocket). The unidirectional valve element is open and allows the passage of air in the absence of a pressure difference between the outside of the dispenser and the internal space of the reservoir, or is configured to open as soon as a low vacuum is generated in the internal space of the reservoir. On the other hand, as soon as the internal space is overpressured compared to the pressure outside the dispenser (atmospheric pressure), the element forming unidirectional valve 22 closes and prevents air from escaping from the space located between the wall 2 and the flexible bag 3.

In particular, the element forming a unidirectional valve 22 can be formed of a thin collar or a flat seal which can either be placed on the outlet of a conduit 25 connecting the outside of the dispenser and the internal space of the reservoir, which closes the conduit 25, or on the contrary become detached or deformed to release the conduit 25, depending on the pressure differential between the inside and the outside of the dispenser. An air-permeable cover as described above can also be used in a variant of this embodiment.

The embodiment of FIG. 6 offers the advantages of that of FIG. 5, and is simple to implement.

Obviously, the different aspects presented above with reference to the figures given by way of example can be combined to form other embodiments of the invention.

The dispenser thus developed allows “multidose” packaging and delivery of the sterile, preservative-free viscous ophthalmic product it contains.

The volume of the bag adapting to that of the viscous ophthalmic product, the entire product can be dispensed without difficulty, including the last doses present in the dispenser, without residual deformation of the wall of the reservoir.

What is claimed is:

1. A dispenser containing an ophthalmic product that can be administered in the form of drips,
the dispenser including a reservoir having a wall defining an internal space and suitable for deforming when pressure is applied to the reservoir by a user of the dispenser and for spontaneously returning to its original shape upon release of thes pressure,
the reservoir further including a flexible bag in the internal space defined by the wall and in which the ophthalmic product is contained, the dispenser including:
an air inlet means for receiving air between the wall of the reservoir and the flexible bag,
a head attached onto a neck of the reservoir and including a drip end piece having a dispensing opening, and
a unidirectional valve interposed between the flexible bag and the dispensing opening, the unidirectional valve being configured to prevent air from outside the dispenser from entering into the flexible bag, such that once the ophthalmic product has been dispensed, the dispensed volume of ophthalmic product is compensated by a corresponding volume of air being received between the wall of the reservoir and the flexible bag via the air inlet means,
wherein the ophthalmic product is a viscous ophthalmic product, that is to say having a viscosity greater than or equal to 200 cP and is sterile and free from preservatives,
and in that, the dispenser including internal surfaces located between the unidirectional valve and the dispensing opening, all of the internal surfaces having a bactericidal treatment and/or a treatment that reduces the wettability thereof.

2. The dispenser containing a viscous ophthalmic product according to claim 1, wherein the flexible bag is formed by delamination of an internal layer of the reservoir.

3. The dispenser containing a viscous ophthalmic product according to claim 1, wherein the flexible bag is a retractable flexible bag.

4. The dispenser containing a viscous ophthalmic product according to claim 1, wherein the air inlet means is formed by a hole in the wall of the reservoir adapted to be closed by a finger of the user.

5. The dispenser containing a viscous ophthalmic product according to claim 1, wherein the air inlet means includes a unidirectional valve, configured to allow receiving air between the wall of the reservoir and the flexible bag and to prevent the air present between the wall of the reservoir and the flexible bag from escaping to the outside of the reservoir.

6. The dispenser containing a viscous ophthalmic product according to claim 1, wherein the bactericidal treatment is based on silver ions or zinc ions.

7. The dispenser containing a viscous ophthalmic product according to claim 6, wherein the bactericidal treatment includes silver ions or zinc ions included in the mass of the elements forming the internal surfaces located between the unidirectional valve and the dispensing opening.

8. The dispenser containing a viscous ophthalmic product according to claim 1, wherein the bactericidal treatment includes a bactericidal surface coating.

9. The dispenser containing a viscous ophthalmic product according to claim 1, wherein an external surface of the unidirectional valve oriented towards the dispensing opening, also has the bactericidal treatment.

10. The dispenser containing a viscous ophthalmic product according to claim 1 wherein the treatment that reduces the wettability of the internal surfaces located between the unidirectional valve and the dispensing opening is a treatment of the fluorinated or silicone plasma treatment type.

11. The dispenser containing a viscous ophthalmic product according to claim 1 wherein the treatment that reduces the wettability of the internal surfaces located between the unidirectional valve and the dispensing opening is a laser surface texturing.

12. The dispenser containing a viscous ophthalmic product according to claim 1 including a removable cap, attached to the dispensing head.

13. The dispenser containing a viscous ophthalmic product according to claim 1 wherein the unidirectional valve includes an element deformable under the effect of excess pressure inside the reservoir of the dispenser, or includes a ball check-valve.

14. The dispenser containing a viscous ophthalmic product according to claim 1, wherein the viscous ophthalmic product includes one or more active ingredients in a therapeutically effective amount selected from the group of anti-glaucoma, anti-inflammatoires, immunosuppressants, antivirals, antibacterials, antifungals or antioxidants, and/or one or more active ingredients in a therapeutically effective amount against dry eyes such as polyacrylic acid derivatives, carbomers, trehalose, hyaluronic acid or its salts, povidone, polyvinyl alcohol, cellulose derivatives, chitosan derivatives, phospholipids, triglycerides.
